(19) United States
(12) Patent Application Publication
(10) Pub. No.: US 2004/0071633 A1
(43) Pub. Date: Apr. 15, 2004

(54) Method for determining efficacy of reverse cholesterol transport enhancing agents

(76) Inventors: Samuel D. Wright, Westfield, NJ (US); Tian-Quan Cai, Hillside, NJ (US)

Correspondence Address: MERCK AND CO INC, P O BOX 2000, RAHWAY, NJ, 070650907

(21) Appl. No.: 10/467,760

(22) PCT Filed: Feb. 19, 2002
(86) PCT No.: PCT/US02/05722

(51) Int. Cl. A61K 49/00; G01N 33/53

(52) U.S. Cl. 424/9.1; 435/7.1

(57) ABSTRACT

This invention provides a method for screening and/or determining the efficacy of known or potential reverse cholesterol transport-enhancing compounds in mammals that avoids the need for measuring cellular protein or mRNA levels and instead employs measurements of CD14 levels in blood.

### BACKGROUND OF THE INVENTION

Reverse cholesterol transport (RCT) is the means by which excess tissue cholesterol is returned to the liver for disposal. It is believed that defects in RCT result in accumulation of cholesterol in tissues in the form of xanthomas and atherosclerotic lesions. It is thus desirable to enhance reverse cholesterol transport in patients with atherosclerosis or xanthomas.

A key gene that promotes RCT is ABCA1 (ABCA1 is also referred to in the published literature as ABC1) (see Mott et al, Atherosclerosis, 152:457-468 (2000)). This gene is believed to function as a “pump” which removes cholesterol from cells to high density lipoprotein (HDL). In keeping with this function in RCT, it has been reported that expression of the ABCA1 gene is increased by cholesterol loading of cells (Langmann et al, Biochem. Biophys. Res. Comm., 257, 29-33 (1999)). Mutations in the ABCA1 gene result in Tangier disease. Cultured cells from patients with Tangier disease are defective in cholesterol efflux (Francis et al. (1995) J. Clin. Invest. 96:78-87), and the patients' condition is marked by massive accumulation of cholesterol in peripheral tissues and very low BDL cholesterol levels.

It is desirable to find treatments capable of enhancing RCT in patients with xanthomas and atherosclerosis. For example, recently it was reported that one of the factors mediating cholesterol-induced upregulation of ABCA1 and RCT is the transcription factor liver X-receptor CXR) (Costet et al (2000), J Biol Chem, 275:28240-2845; Schwartz et al (2000), Biochem Biophys Res Comm, 274:794-802). This protein senses excess intracellular cholesterol levels, and initiates gene expression changes that promote reverse cholesterol transport. LXR is thought to upregulate a large number of genes that all serve to promote RCT. In addition to upregulating ABCA1, LXR is believed to upregulate genes including ABCA8, an additional “pump” protein, and CETP, a protein that moves cholesterol from HDL to other lipoproteins. Therefore, it has been found that ligands of LXR, including ligands of both LXRα and LXRβ, can be useful as drugs to increase the expression of ABCA1, increase levels of HDL and thereby decrease the risk of atherosclerosis, myocardial infarction and related conditions such as peripheral vascular disease and ischemic stroke. Other factors that act to upregulate the proteins needed for RCT are also the subject of ongoing research.

However, since most known participants in RCT are intracellular proteins, the only method currently available for determining the efficacy of a known or potential RCT-enhancing drug is to measure cellular protein or mRNA levels. The need for cells such as liver or spleen cells for these type of measurements precludes routine use of such assays in humans. Therefore there remains a need for providing a noninvasive and efficient method for determining the efficacy of a known or potential RCT-enhancing agent in mammals, particularly in humans.

CD14 is a 55 kDa protein expressed as both a soluble protein in plasma (sCD14) and as a membrane protein on the surface of monocytes and macrophages (mCD14) (Wright, S. D. (1999), Innate recognition of microbial lipids, Inflammation: Basic Principles and Clinical Correlates, 3rd ed., J. I. Gallin, and R. Snyderman eds. Raven Press, New York, N.Y.). Though first characterized as a binding protein for bacterial lipopolysaccharide (LPS), it has since been demonstrated that sCD14 binds a broad range of phospholipids and can shuttle these lipids into and out of membranes (Yu, B., Hailman, E., and Wright, S. D. (1997) J. Clin. Invest. 99, 315-324; Vasselon, T., Pironkova, R., and Detmers, P. A. (1997) J. Immunol. 159, 4498-4505; Vasselon, T., Hailman, E., Thieringer, R., and Detmers, P. A. (1999) J. Exp. Med. 190, 509-521; Wurfel, M. M., Hailman, E., and Wright, S. D. (1995) J. Exp. Med. 181,1743-1754; and Detmers, P. A., Thieblemont, N., Vasselon, T., Pironkova, R., Miller, D. S., and Wright, S. D. (1996) J. Immunol. 157, 5589-5596).

The shuttle function of CD14 is illustrated by the finding that the rate of transport of insoluble LPS into HDL particles (Wurfel, et al, ibid.) or cells (Hailman, E., Vasselon, T., Kelley, M., Busse, L. A., Hu, M. C., Lichenstein, H. S., Detmers, P. A., and Wright, S. D. (1996) J. Immunol. 156, 4384-4390) is dramatically enhanced by the addition of sCD14. Kinetic analysis of this facilitated transport shows that the LPS is first bound to sCD14, and the bound lipid is then surrendered to the HDL particle (Wurfel, et al, ibid.).

The plasma membrane contains phospholipid and cholesterol in approximate molar equivalence, and cells are known to export these two lipids together (Oram, J. F., and Yokoyama, S. (1996) J. Lipid Res. 37, 2473-2491). However, it was previously unknown if CD14 was involved in facilitating export of cholesterol from cells.

We have now discovered that the protein CD14 participates in RCT, and that the CD14 gene is upregulated by agents that increase RCT. Levels of CD14, both soluble and membrane-bound, thus provide a measure for determining the efficacy of known or potential RCT-enhancing agents.

### SUMMARY OF THE INVENTION

One object of the present invention is to provide a method for determining the efficacy of a known or potential reverse cholesterol transport-enhancing agent in mammals, particularly humans, that avoids the need for measuring cellular protein or mRNA levels and instead employs blood level measurements of CD14 levels.

Accordingly, this invention provides a method for evaluating the efficacy of a reverse cholesterol transport-enhancing test agent in a mammal comprising: measuring the level of CD14 protein present in a standard sample of the mammal's blood to obtain one CD14 protein measurement; measuring the level of CD14 protein present in a sample of the mammal's blood after administration of the test agent to obtain a second CD14 protein measurement; and comparing the two CD14 protein measurements to determine if the measurement obtained after test agent administration is greater than the measurement obtained from the standard blood sample; and wherein the CD14 protein is selected from sCD14 and mCD14 and is the same for both measurements.

Additional objects and embodiments of this invention will be evident from the following detailed description.

### BRIEF DESCRIPTION OF THE DRAWINGS

FIGS. 1a, 1b and 1c are graphs showing the effect of sCD14 concentration on cholesterol efflux. [³H]cholesterol-labeled THP-1 cells were incubated with the indicated concentration of sCD14 (closed circles), apo A-I (open circles) or HSA (inverted triangles) in RPMI for 30 min (a and b) or for 20 h (c). Each point represents the mean of three measurements±SD (a) or the average of duplicate samples±range (b and c). Data are representative of five independent experiments with similar results.

FIG. 2 is a graph showing the kinetics of cholesterol efflux by sCD14 and/or apo A-I. [³H]cholesterol-labeled THP-1 cells were incubated with or without 5 μg/ml of sCD14 and/or 100 μg/ml of apo A-I in RPMI. Background efflux without proteins at each time was measured and subtracted from other data point. Closed circles, sCD14; open circles, apo A-I; inverted triangles, sCD14 and apo A-L. Data represents the mean of three measurements±SD. The data were repeated with similar results.

FIG. 3 is a graph showing the dose dependent additive effect of sCD14 for cholesterol efflux to apo A-I but not to HDL. [³ H]cholesterol-labeled TBP-1 cells were incubated in buffer containing 50 μg/ml of apo A-I, HDL3 or HDL with or without the indicated concentration of sCD14 for 1 h. Open bar, without sCD14; hatched bar, 10 μg/ml sCD14; black bar, 25 μg/ml sCD14. Background efflux without proteins at each time was measured and subtracted from other data point. Asterisks show statistical significance compared to the data without sCD14: *, p<0.01; **, p<0.001. Data are representative of three independent experiments with similar results.

FIGS. 4a and 4b are graphs showing inhibition for apo A-I-dependent cholesterol efflux by various anti-CD14 antibodies. [³H]cholesterol-(a) or [³H]choline-(b) labeled THP-1 cells were incubated with or without apo A-I and/or various anti-CD14 antibodies for 20 h. Antibodies are indicated at the bottom of the bars. Antibody concentrations were 10 μg/ml for mAb and 100 μg/ml for polyclonal antibody (a and b). Asterisks show statistical significance (p<0.01) compared to the data without anti-CD14. Data are representative of three independent experiments with similar results.

FIGS. 5a and 5b are graphs showing that activation of LXR increases mRNA expression of ABCA1 and CD14. THP-1 or HepG2 cells (5×10⁵ cells/well) were plated in 6-well plates, and mixed with buffer (open bar) or 22(R)—OH cholesterol (10 μM, closed bar). After overnight incubation (˜16 hr) at 37° C., RNA samples were prepared from the cultured cells, and mRNAs for ABCA1 (a) and CD14 (b) were measured as described in Example 4, below. Results are presented as fold of control (untreated cells), and data are shown as the means±range of duplicate determinations.

### DETAILED DESCRIPTION OF THE INVENTION

We have discovered that the protein CD14 participates in RCT. CD14 is expressed both as a soluble protein in plasma (sCD14) and as a membrane protein on the surface of monocytes (mCD14). Addition of sCD14 to cells enhances cellular efflux of cholesterol to apolipoprotein A-I (apoAI), and blockade of mCD14 with antibodies blocks cellular efflux of cholesterol. Consistent with this function, we have observed that addition of 22 hydroxycholesterol, a ligand for IXR and a compound known to enhance cholesterol efflux from cells, causes coordinate upregulation of not only ABCA1 but also CD14 mRNA. As a result, measurements of blood levels of CD14 can be used to provide a measure of efficacy of RCT-enhancing agents.

Furthermore, CD14 is readily assayed in plasma and it is stable. sCD14 can be measured by EUSA (enzyme-linked immunosorbent assay) on plasma, and mCD14 can be measured by FACS (fluorescent activated cell sorting) on blood monocytes. Assays for measuring blood levels of sCD14 and mCD14 are described in the published literature. For example, an ELISA assay for measuring sCD14 is described in Detmers P A, Zhou D, Powell D, Lichenstein H, Kelley M, Pironkova R. Endotoxin receptors (CD14) are found with CD16 (Fc gamma RIII) in an intracellular compartment of neutrophils that contains alkaline phosphatase, J Immunol. 1995 Aug. 15;155(4):2085-95, herein incorporated by reference. There are also commercially available ELISA kits for measuring sCD14, such as one available for measuring soluble human CD14 from R & D Systems Inc., 614 McKinley Place N.E., Minneapolis, Minn. 55413 (current catalog number is DC140).

Several assays for measuring blood levels of mCD14 are described in the published literature, including for example, as described in Patino R, Ibarra J, Rodriguez A, Yague M R, Pintor E, Femandez-Cruz A, Figueredo A., Circulating monocytes in patients with diabetes mellitus, arterial disease, and increased CD14 expression, Am J Cardiol. 2000 Jun. 1; 85(11):1288-91; and Wright et al (1986), J Exp. Med, 163:1245-1259, both herein incorporated by reference.

This invention provides a method for evaluating the efficacy of a known or potential reverse cholesterol transport-enhancing agent, herein referred to as a test agent, in a mammal comprising measuring the level of CD14 protein present in a standard sample of the mammal's blood to obtain one CD14 protein measurement; measuring the level of CD14 protein present in a sample of the mammal's blood after administration of the test agent to obtain a second CD14 protein measurement; and comparing the two CD14 protein measurements to determine if the measurement obtained after test agent administration is greater than the measurement obtained from the standard blood sample; and wherein the CD14 protein is selected from sCD14 and mCD14 and is the same for both measurements.

Measurement of blood levels of CD14 is required to practice the instant invention. Either sCD14 or mCD14 may be measured, but comparisons are intended to be made between or among measurements of the same form of CD14, that is measurements of sCD14 are to be compared against other measurements of sCD14, and measurements of mCD14 are to be compared against other measurements of mCD14.

The agent which is being evaluated for its efficacy at enhancing RCT will be referred to herein as the test agent. The test agent can be any agent, chemical or non-chemical, desired to be and capable of being tested with a mammal. The term “test agent” encompasses both known RCT-enhancing agents, such as LXR ligands, and potential RCT-enhancing agents whose activity for enhancing RCT is not yet known or confirmed. For example, the term ‘test agent’ is intended to include not only compounds, mixtures of compounds and pharmaceuticals, but also vitamins and consumable dietary items such as foods and food supplements. The term test agent further includes known and potential physical and behavioral inducers of RCT enhancement such as physical exercise and/or weight-loss dieting. The tested physical and behavioral inducers of RCT enhancement may also be administered in combination with a drug therapy, for example weight-loss dieting along with administration of a weight-loss drug. Therefore, the instant method invention can be used, for example, to screen agents such as compounds, foods, vitamins and/or different types of physical and behavioral activities for their RCT-enhancing activity, as well as to evaluate the relative activity of known RCT-enhancing agents. The instant invention is not limited by any particular type of agent that is or can be tested; rather, the invention encompasses the use of CD14 measurements as a biological marker for indicating whether or not enhancement of RCT has occurred.

The blood sample may be taken at any time of choice after the start of test agent administration. Persons skilled in the art are capable of determining a desired length of time for administration of the test agent before the mammal's blood sample is taken, which may vary depending on the chosen test agent or combination of agents and the objective of the experiment. For example, since HMG-CoA reductase inhibitors take about 2 weeks to exert their LDL lowering effect, a blood sample could be taken 2-4 weeks after the start of a daily MG-CoA reductase inhibitor drug therapy; or the blood sample could be taken immediately following a physical exercise workout or a meal, or after a period of weeks or months of following an exercise or weight-loss dietary regimen. Blood samples could be taken at a variety of times during the administration of a test agent to determine when, if at all, an RCT-enhancement occurs.

In order to evaluate the efficacy of the test agent, the results from the CD14 blood measurement must be compared to the results of a standard blood sample from the mammal. As intended herein, the “standard” blood sample includes both a sample obtained absent administration of the test agent to the mammal, as well as a sample obtained after administration to the mammal of a known RCT-enhancing agent which is different from the test agent. When the test agent is a known or potential physical or behavioral inducer of RCT enhancement, the standard blood sample may be obtained absent the physical or behavioral inducer (for example, a blood sample from a rested state or before the start of a weight-loss diet) or it could be obtained after a different type of physical or behavioral activity for comparison of results, such as a standard blood sample obtained after physical exercise or a weight-loss diet that is different from the tested agent.

When the standard blood sample is obtained after administration of a known RCT-enhancing agent, the known RCT-enhancing agent, also referred to herein as a standard RCT-enhancing agent, may be used as a standard agent against which one or more test agents are compared for efficacy. The practice of this invention requires that when a standard RCT-enhancing agent is administered to obtain a standard blood sample, then the test agent must be a different agent than the standard RCT-enhancing agent that is administered to obtain the standard blood sample.

The order in which the test blood sample and the standard blood sample are obtained and measured is a matter of choice and is not intended to be limited to any particular order.

Accordingly, one embodiment of this invention is a method for evaluating the efficacy of a reverse cholesterol transport-enhancing test agent in a mammal comprising:

measuring the level of CD14 protein present in a sample of the mammal's blood absent administration of the test agent to obtain one CD14 protein measurement;

measuring the level of CD14 protein present in a sample of the mammal's blood after administration of the test agent to obtain a second CD14 protein measurement; and

comparing the two CD14 protein measurements to determine if the measurement obtained after test agent administration is greater than the measurement obtained absent test agent administration;

and wherein the CD14 protein is selected from sCD14 and mCD14 and is the same for both measurements.

Another embodiment of this invention is a method for determining relative efficacy for a reverse cholesterol transport-enhancing test agent in a mammal comprising:

measuring the level of CD14 protein present in a sample of the mammal's blood after administration of a standard RCT-enhancing agent to obtain one CD14 protein measurement;

measuring the level of CD14 protein present in a sample of the mammal's blood after administration of the test agent to obtain a second CD14 protein measurement; and

comparing the two CD14 protein measurements to determine the relative efficacy of the test agent as compared to that of the standard RCT-enhancing agent;

and wherein the CD14 protein is selected from sCD14 and mCD14 and is the same for both measurements.

The methods of this invention are useful for evaluating efficacy of any agents having reverse cholesterol transport-enhancing activity and those agents which are desired to be screened for such activity. Examples of classes of agents that could be screened include but are not limited to compounds such as L ligands, human peroxisome proliferator activated receptor (PPAR) agonists, including agonists of PPARδ, PPARβ and PPARγ as well as agonists having activity for one or more of the PPAR subtypes, 3-hydroxy-3-methylglutaryl coenzyme A (HMG-CoA) reductase inhibitors, acyl-coenzyme A:cholesterol acyltransferase (ACAT) inhibitors including ACAT-1 (sometimes referred to as ACAT I), described in U.S. Pat. No. 5,834,283, and ACAT-2 (sometimes referred to as ACAT II), described in WO 97/45439, fish oils, vitamins such as nicotinic acid (vitamin B3), niacinamide and analogs thereof, and alcohols such as ethanol.

The term LXR includes all subtypes of this receptor and corresponding genes which encode such subtypes. Specifically LXR includes LXRα and LXRβ, and ligands of LXR include agonists and antagonists of LXRα and/or LXRβ. LXRα has been referred to under a variety of names and for purposes of this application LXRα should be understood to mean any gene referred to as LXRα, LXRₐ, LXRalpha, RID-1, NR1H3 or a gene with homology to accession number U22662 or a protein with homology to a protein encoded by such a polynucleotide. Similarly, LXRβ should be understood to include any gene referred to as LXR_{b}, LXRβ, LXRbeta, NER, NER1, UR, OR-1, R1P15, NR1H2 or a gene with homology to accession number U07132 or a protein with homology to a protein encoded by such a polynucleotide. LXR ligands can be identified by published procedures such as described in Zhou et al (1998), Molecular Endo, 12:1594-1604, herein incorporated by reference. LXR ligands include compounds such as 22(R)-hydroxycholesterol, 20(S)-hydroxycholesterol and 25-hydroxycholesterol, all well known for their LXR activity.

PPAR agonists are well known in the art and can be identified by known assays. Examples of PPARγ agonists include compounds commonly referred to as glitazones, for example troglitazone, pioglitazone and rosiglitazone, as well as those compounds included within the structural class known as thiazolidinediones, in addition to those PPARγ agonists outside the thiazolidinedione structural class. PPARα agonists include, for example the fibrate class of compounds such as clofibrate, fenofibrate and gemfibrozil. PPARγ and PPARα agonists can be identified, for example, by the assay described in. Zhou, et al (1998), Molecular Endo, 12:1594-1604, herein incorporated by reference. PPARδ agonists can be identified, for example, by the assays described in WO97/28149 published Aug. 7, 1997, herein incorporated by reference, which also provides examples of PPARδ agonists.

Compounds that have inhibitory activity for HMG-CoA reductase can be readily identified using assays well known in the art. For example, see the assays described or cited in U.S. Pat. No. 4,231,938 at col. 6, and WO 84/02131 at pp. 30-33, herein incorporated by reference. HMG-CoA reductase inhibitors include but are not limited to compounds known as “statins” which are part of a structural class of compounds that contain a moiety which can exist as either a 3-hydroxy lactone ring or as the corresponding 3,5-dihydroxy open-acid. HMG-CoA reductase inhibitors encompass statins in their lactonized and their dihydroxy open acid forms and pharmaceutically acceptable salts and esters thereof. Examples of HMG-CoA reductase inhibitors of the statin class include but are not limited to lovastatin (see U.S. Pat. No. 4,342,767); simvastatin and dihydroxy open-acid simvastatin (see U.S. Pat. No. 4,444,784); pravastatin (see U.S. Pat. No. 4,346,227); fluvastatin (see U.S. Pat. No. 5,354,772); atorvastatin (see U.S. Pat. No. 5,273,995); cerivastatin (see U.S. Pat. No. 5,177,080), NK-104 (see U.S. Pat. No. 5,856,336), ZD-4522 (U.S. Pat. No. 5,260,440) and pharmaceutically acceptable salts and esters thereof.

Compounds which have inhibitory activity for ACAT, including those which selectively inhibit ACAT-1 or ACAT-2, and those which have dual inhibitory activity for both ACAT-1 and ACAT-2, can be readily identified using assays well-known in the art, for example as described in Chang C. C., Lee C. Y., Chang, E. T., Cruz, J. C., Levesque, M. C., Chang, T. Y.: J. Biol. Chem. 273:35132-35141 (1998), Recombinant acyl-CoA:cholesterol acyltransferase-1 (ACAT-1) purfied to essential homogeneity utilizes cholesterol in mixed micelles or in vesicles in a highly cooperative manner, herein incorporated by reference. For example, ACAT inhibitors include but are not limited to compounds described in U.S. Pat. Nos. 5,120,738; 5,340,807; 5,475,130; 5,668,136; 5,760,087; and additionally described in published patent applications WO96/26925; WO97/16184; EP 0 635 501 A1 European Application No. 94305305.8) Sliskovic, D. R., CI-1011: An a typical ACAT inhibitor with antiatherosclerotic activity, Proceedings, XIVth International Symposium on Medicinal Chemistry, F. Awouters (editor) Elsevier Science B. V., 433-441 (1997); and Tanaka, A. et al., Inhibition of acyl-CoA:cholesterol O-acyltransferase. 2. Identification and structure-activity relationship of a novel series of N-alkyl-N-(heteroaryl-substituted benzyl)-N′-arylureas, J. Med. Chem., 41:2390-2410 (1998).

Cellular export of phospholipids to apo A-I is accompanied by export of cellular cholesterol. In order to determine whether sCD14 also facilitates cholesterol efflux from cells, the following experiments were performed.

### EXAMPLE 1

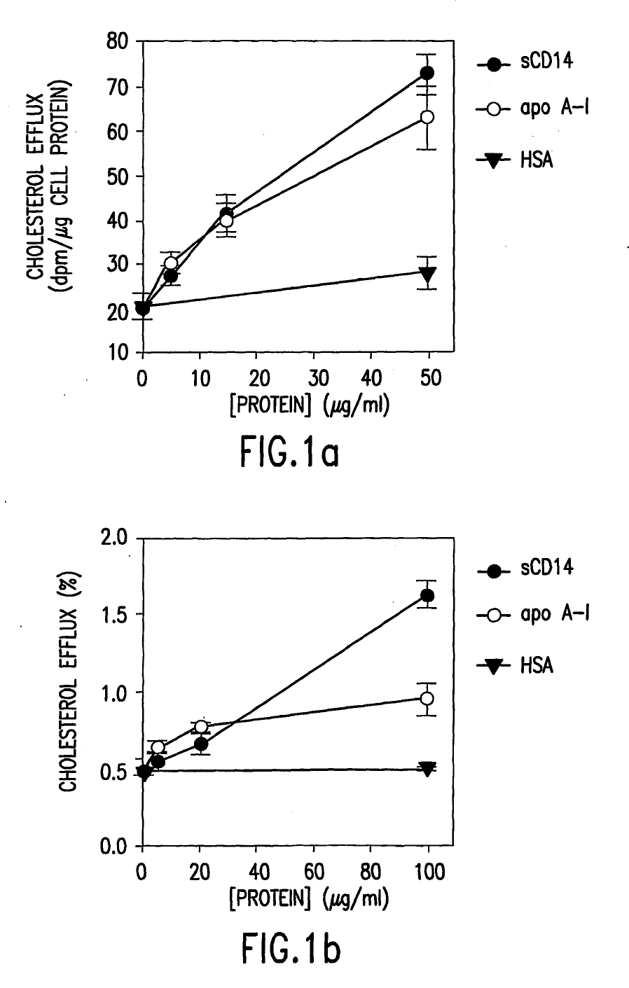

sCD14 Mediation of Cholesterol Efflux from Cells

[³H]cholesterol-labeled THP-1 cells were incubated with increasing concentrations of sCD14, apo A-I or the control protein, HSA, and radioactivity in the supernatant was determined. The initial rate of cholesterol efflux was explored in studies of short duration (30 min, FIG. 1a and with extended dose response in FIG. 1b). We were surprised to find that sCD14 strongly potentiated cholesterol efflux with a potency similar to that of apo A-I in 30 min assays. While the efflux of cholesterol to apo A-I is saturable at 10-20 μg/ml (FIGS. 1a and 1b), CD14-mediated cholesterol efflux in 30 min assays did not show saturation even up to 100 μg/ml (FIG. 1b). Because of the linear dose-dependence of sCD14-mediated efflux and because no other cholesterol acceptor was present in the incubation, these observations appear most consistent with a direct, low affinity binding of cholesterol to sCD14.

The maximal extent of cholesterol efflux mediated by apo A-I and sCD14 was explored in parallel experiments of 24 hr duration (FIG. 1c). Under these conditions, as little as 10 μg/ml apo A-I led to near maximal cholesterol efflux, and this amount of efflux far exceeded that produced by comparable amounts of sCD14. Comparison of dose-dependence curves in FIG. 1c showed that apo A-I could mediate cholesterol efflux with a capacity 10-20-fold higher than that of CD14. These data suggest that sCD14 by itself may facilitate cholesterol efflux from cells. However, because of the limiting concentrations of sCD14 in plasma (3-5 μg/ml) and the far greater concentration of apo A-I (˜1 mg/ml), sCD14 per se is unlikely to be a significant reservoir or destination for the cholesterol in plasma.

### EXAMPLE 2

sCD14 Accelerates Cholesterol Efflux to Apo A-I

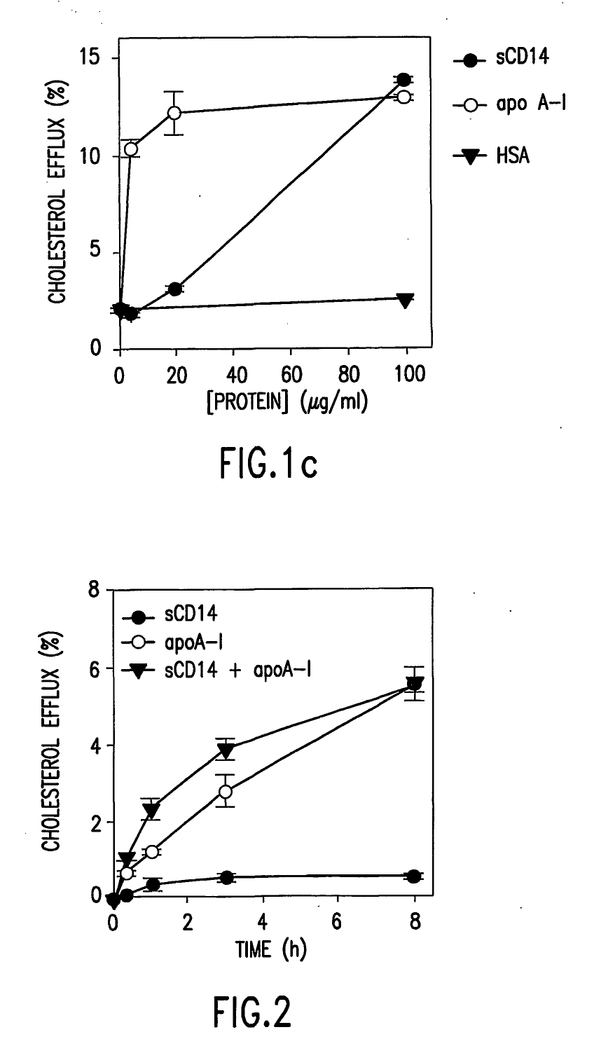

The following experiment was performed to determine if sCD14 could facilitate the efflux of cholesterol to its major acceptor, apo A-I (FIG. 2). For these studies, we chose a concentration of sCD14 similar to that in plasma (5 μg/ml), a concentration that mediates only modest cholesterol release. Kinetic analysis (FIG. 2) showed the expected low efflux mediated by sCD14 alone but a larger efflux to apo A-I. However, we observed a clear acceleration of efflux when CD14 and apo A-I were combined. In the first hour, addition of sCD14 caused clear enhancement of the efflux rate to apo A-I, in spite of a high concentration of apo A-I (100 μg/ml) that saturates CD14-independent cholesterol efflux. The enhancement continued at least up to 3 hours. These data suggest that sCD14 may make important contributions to the rate of cholesterol efflux from cells. It should be noted that the enhancement of efflux by sCD14 was only seen early in the time course and was no longer apparent at 8 hours. These data suggest that sCD14 may hasten the movement of cholesterol to its destination, but sCD14 itself is not itself a significant destination for cholesterol.

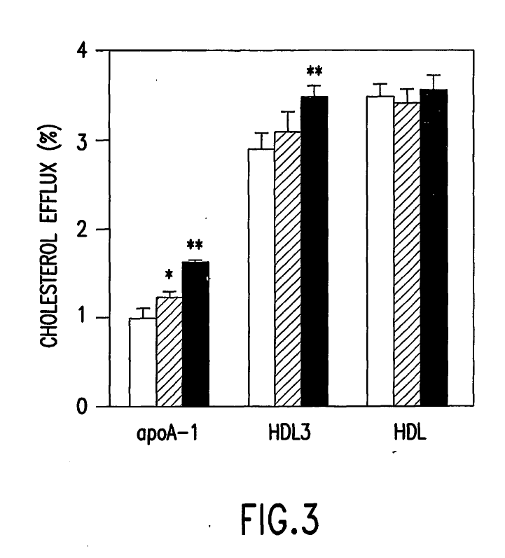

Cholesterol efflux to apo A-I at the one hour time point was further studied in FIG. 3. These data confirm the acceleration of efflux caused by sCD14 and show dose dependence. Additional studies showed that sCD14 also enhanced efflux in the presence of the dense, lipid-poor subspecies HDL3 but failed to significantly enhance efflux to plasma HDL.

### EXAMPLE 3

mCD14 Plays a Role in Lipid Efflux to Apo A-I

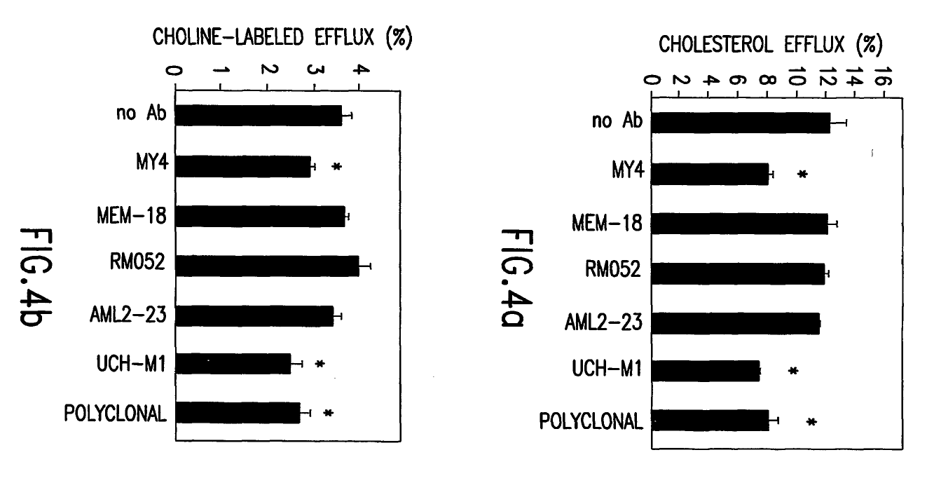

The above studies on the role of sCD14 employed a macrophage-like cell line (THP-1) which expresses mCD14. To determine if mCD14 also contributes to lipid export to apo A-I, monoclonal and polyclonal anti-CD14 antibodies were incubated with THP-1 cells, and apo A-I-dependent lipid efflux was measured. As shown in FIG. 4a, two anti-CD14 mAb, MY4 and UCH-M1, and polyclonal anti-CD14 each inhibited cholesterol efflux from THP-1 cells to apo A-I by 34-39%. Parallel studies showed similar but somewhat weaker inhibition of phospholipid efflux by these antibodies (FIG. 4b). The inhibition by anti-CD 14 mAbs appears specific since antibodies against other antigens (CD11b, CD55) had no effect in this assay. Moreover, the inhibition appeared specific for particular epitopes of CD14, and antibodies against other epitopes of CD14 (MEM18, RM052 and AML2-23) showed no inhibition under our conditions (FIG. 4a and 4b).

### EXAMPLE 4

Regulation of CD14 by LXR

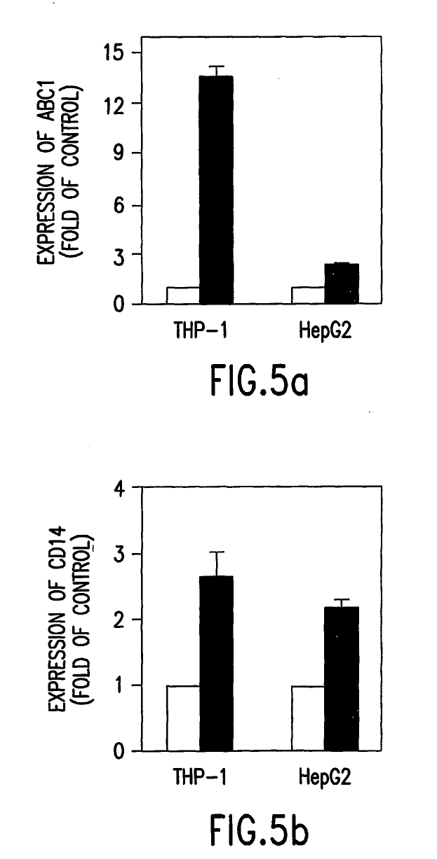

Recent studies indicate that cholesterol efflux from cells is regulated by the transcription factor, LXR, which binds regulatory oxysterols and enhances expression of ABCA1 (see Costet, P., Luo, Y., Wang, N., and Tall, A. R. (2000) J. Biol. Chem. 275, 28240-28245; and Schwartz, K., Lawn, R. M., and Wade, D. P. (2000) Biochem. Biophys. Res. Commun. 274, 794-802.). If CD14 shared with ABCA1 a role in cholesterol efflux from cells, the expression of CD14 might be regulated in parallel with ABCA1 to help achieve cholesterol homeostasis. To test this, cells were incubated with 22(R)-hydroxycholesterol (22R—OH cholesterol), a specific ligand for LXR, and expression of ABCA1 and CD14 mRNA was measured by real time PCR. As shown in FIG. 5, addition of 22R—OH cholesterol to either THP-1 monocytes or the hepatoma line, HepG2, led to a consistent rise not only in ABCA1 (FIG. 5a) but also in CD14 expression (FIG. 5b). The dose dependence for induction of ABCA1 and CD14 was roughly similar with half-maximal induction observed at ˜10 μM (not shown). Induction of ABCA1 appeared slightly more rapid than that of CD14, but both reached a plateau level by 16 hr. The magnitude of the response of CD14 to 22R—OH cholesterol was less than that observed for ABCA1, but the increase was consistently seen with either of these two cell types. The observation of parallel regulation of ABCA1 and CD14 is consistent with a shared function of these two proteins in cholesterol efflux.

Real-Time Quantitative PCR Analysis of mRNA Expression

Real-time quantitative TaqMan PCR analysis (Heid, C. A., Stevens, J., Livak, K. J., and Williams, P. M. (1996) Genome Res. 6, 986-994) was used to determine the relative levels of CD14 and ABCA1 mRNA. RT-PCR and TaqMan PCR reactions were performed according to the manufacturer's instructions (PE Biosystems, Norwalk, Conn., TaqMan Gold RT-PCR protocol and TaqMan Universal PCR Master Mix). Sequence-specific amplification was detected with an increased fluorescent signal of FAM (reporter dye) during the amplification cycle. Amplification of the gene for human GAPDH was performed in the same reaction on all samples tested as an internal control for variations in RNA amounts. CD14 and ABCA1 mRNA was subsequently normalized to GAPDH mRNA levels. mRNA levels were presented as fold difference of treated cells against untreated cells.

What is claimed is:

1. A method for evaluating the efficacy of a reverse cholesterol transport-enhancing test agent in a mammal comprising
measuring the level of CD14 protein present in a standard sample of the mammal's blood to obtain one CD14 protein measurement;
measuring the level of CD14 protein present in a sample of the mammal's blood after administration of the test agent to obtain a second CD14 protein measurement; and
comparing the two CD14 protein measurements to determine if the measurement obtained after test agent administration is greater than the measurement obtained from the standard blood sample;
and wherein the CD14 protein is selected from sCD14 and mCD14 and is the same for both measurements.

2. The method of claim 1 wherein the test agent is a potential RCT-enhancing agent.

3. The method of claim 1 wherein the test agent is a known RCT-enhancing agent.

4. The method of claim 1 wherein the standard blood sample is a sample of the mammal's blood absent administration of the test agent.

5. The method of claim 1 wherein the standard blood sample is a sample of the mammal's blood after administration of a known RCT-enhancing agent.

6. A method for determining relative efficacy for a reverse cholesterol transport-enhancing test agent in a mammal comprising:
measuring the level of CD14 protein present in a sample of the mammal's blood after administration of a standard RCT-enhancing agent to obtain one CD14 protein measurement;
measuring the level of CD14 protein present in a sample of the mammal's blood after administration of the test agent to obtain a second CD14 protein measurement; and
comparing the two CD14 protein measurements to determine the relative efficacy of the test agent as compared to that of the standard RCT-enhancing agent;
and wherein the CD14 protein is selected from sCD14 and mCD14 and is the same for both measurements.

7. The method of claim 6 wherein the test agent is a potential RCT-enhancing agent.

8. The method of claim 6 wherein the test agent is a known RCT-enhancing agent.

9. The method of claim 1 wherein the test agent is an LXR ligand.

10. The method of claim 1 wherein the test agent is a PPAR agonist.

11. The method of claim 1 wherein the test agent is an HMG-CoA reductase inhibitor.

12. The method of claim 1 wherein the test agent is a fish oil.

13. The method of claim 1 wherein the test agent is an alcohol.

14. The method of claim 1 wherein the test agent is a vitamin.

15. The method of claim 14 wherein the test agent is niacin, niacinamide or an analog thereof.

16. The method of claim 1 wherein the test agent is an ACAT inhibitor.

17. The method of claim 1 wherein the test agent is a weight loss drug.

18. The method of claim 1 wherein the test agent is a known or potential physical inducer of RCT enhancement.

19. The method of claim 1 wherein the test agent is a known or potential behavioral inducer of RCT enhancement.

20. The method of claim 1 wherein the mammal is a human.
